(19) United States
(12) Patent Application Publication
(10) Pub. No.: US 2020/0187894 A1
(43) Pub. Date: Jun. 18, 2020

(54) Diaphragm, Methods for Manufacturing Same and Stethoscope Provided with Same

(71) Applicant: Vitalchains Corporation, Menlo Park, CA (US)

(72) Inventors: Tzu-Chih Lin, Chupei City (TW); Yu-Cheng Huang, Chupei City (TW)

(21) Appl. No.: 16/644,105

(22) PCT Filed: Sep. 6, 2018
(86) PCT No.: PCT/US2018/049750

§ 371 Date: Mar. 3, 2020

(30) Foreign Application Priority Data
Sep. 6, 2017 (CN) 201710795321.9
Sep. 6, 2017 (CN) 201721135206.0
Sep. 6, 2017 (TW) 106130520

(51) Int. Cl. A61B 7/04

(52) U.S. Cl. CPC A61B 7/04

(57) ABSTRACT

The present invention relates to a diaphragm, and stethoscopes provided with the same. The diaphragm having a plate-shaped vibratable portion, and the vibratable portion has a first surface. The diaphragm further comprises a compliant structural layer disposed on the first surface. When the diaphragm is assembled with a sound collecting head, the compliant structural layer is in contact with the sound collecting head to enhance the airtightness between the diaphragm and the sound collecting head to prevent the acoustic vibration energy from dissipating. The invention also relates to methods for manufacturing the diaphragm which can quickly produce a large number of diaphragms by simple processes.

### FIELD OF THE INVENTION

The present invention relates to a diaphragm which can reduce the loss of acoustic vibration energy and, in particular, can improve the quality of auscultation, as well as a stethoscope provided with the diaphragm. The invention further relates to methods for preparing the aforementioned diaphragm.

### BACKGROUND OF THE INVENTION

For a medical care practitioner who intends to effectively diagnose the health of internal organs in a patient's body, they may use a stethoscope to attach to the chest or abdomen of the patient, allowing the sound waves generated by the internal organs to be transmitted to the ears of the medical care practitioner. The stethoscope is a fast, safe and portable diagnostic tool for monitoring the symptoms of heart, lung and gastrointestinal diseases.

At present, the stethoscopes used by medical care personnel may be roughly categorized into two types, namely, single-sided stethoscopes and double-sided stethoscopes. Both types of stethoscopes include an auscultation head composed of a diaphragm and a sound collecting head for collecting and amplifying sound, and ear tubes and earplugs through which the sound is transmitted to the ears of the wearer. Therefore, the auscultation head that collects sound is an important and indispensable part for performing the function of the stethoscope. It is known that the airtightness of the auscultation head is a major factor that affects the auscultation quality. However, it is inevitable for a conventional auscultation head to have a gap between the diaphragm and the sound collecting head, resulting in a poor airtight sealing effect which in turn deteriorates the dissipation of acoustic vibration energy and the sound attenuation rate and reduces the auscultation quality.

### SUMMARY OF THE INVENTION

In view of this, the invention provides a diaphragm adapted for assembly with a sound collecting head, which can increase the airtight sealing effect between the two parts and reduce the loss of acoustic vibration energy.

In a first aspect provided herein is a diaphragm comprising:

a vibratable portion having a first surface; and

a compliant structural layer disposed on the first surface.

In a second aspect provided herein is a stethoscope, comprising:

a sound receiving unit for receiving sound, comprising a sound collecting head and the diaphragm stated above, with the compliant structural layer of the diaphragm being attached to the sound collecting head; and a sound transmission unit connected to the sound receiving unit for transmitting the sound.

In a preferred embodiment, the compliant structural layer has a thickness between 0.04 mm and 0.1 mm. In a more preferred embodiment, the thickness of the compliant structural layer is between 0.04 mm and 0.08 mm.

In a preferred embodiment, the first surface is coated entirely with the compliant structural layer. In another preferred embodiment, the compliant structural layer is disposed on an outer periphery of the first surface.

In a preferred embodiment, the compliant structural layer is formed by curing a compliant material selected from the group consisting of silicones, soft touch paints, thermoplastic polyurethanes and thermoplastic rubbers.

In a preferred embodiment, the stethoscope further comprises a retaining ring for clamping an outer periphery of the diaphragm and the sound collecting head, so as to retain the diaphragm on the sound collecting head.

In another preferred embodiment, the sound collecting head is formed with a groove that is configured to be complementary in shape to the retaining ring.

In a preferred embodiment, the diaphragm further comprises a retaining portion extending from an outer periphery of the vibratable portion and protruding from the first surface, and the diaphragm is retained by the retaining portion on the sound collecting head.

In another preferred embodiment, the retaining portion is further bent inwardly to constitute a flange, and the diaphragm is retained by the flange on the sound collecting head.

In a third aspect provided herein is a method for manufacturing the above-mentioned diaphragm, comprising in sequence the steps of:

providing a membrane having a first surface;

spraying a compliant material on the first surface of the membrane;

curing the compliant material to form a compliant structural layer; and

stamping the membrane coated with the compliant structural layer to form a plurality of diaphragms.

In a fourth aspect provided herein is a method for manufacturing the above-mentioned diaphragm, comprising in sequence the steps of:

providing a membrane;

preforming and stamping the membrane to form a plurality of semi-finished diaphragms, wherein each of the semi-finished diaphragms comprises a vibratable portion having a first surface, and a retaining portion formed at an outer periphery of the vibratable portion and protruding from the first surface of the vibratable portion;

spraying a compliant material on the first surface of the vibratable portion; and

curing the compliant material to form a compliant structural layer, thereby forming the diaphragms, each having the retaining portion.

### BRIEF DESCRIPTION OF THE DRAWINGS

The above and other objects, features and effects of the invention will become apparent with reference to the following description of the preferred embodiments taken in conjunction with the accompanying drawings, in which:

FIG. 1 is a perspective view of the diaphragm according to the first embodiment of the invention;

FIG. 2 is a sectional exploded view of the diaphragm and the sound collecting head according to the first embodiment of the invention;

FIG. 3 is a partial cross-sectional side view of the diaphragm and the sound collecting head according to the first embodiment of the invention;

FIG. 4 is a cross-sectional exploded view of the diaphragm and the sound collecting head according to the second embodiment of the invention;

FIG. 5 is a cross-sectional exploded view of the diaphragm and the sound collecting head according to the third embodiment of the invention;

FIG. 6 is a schematic flowchart showing the manufacturing of the diaphragm according to the first embodiment of the invention;

FIG. 7 is a schematic flowchart showing the manufacturing of the diaphragm according to the second embodiment of the invention; and

FIG. 8 is a graph showing attenuation versus frequency for the respective embodiments according to the invention and a comparative example provided herein.

### DETAILED DESCRIPTION OF PREFERRED EMBODIMENTS

Unless specified otherwise, the following terms as used in the specification and appended claims are given the following definitions. It should be noted that the indefinite article “a” or “an” as used in the specification and claims is intended to mean one or more than one, such as “at least one,” “at least two,” or “at least three,” and does not merely refer to a singular one. In addition, the terms “comprising/comprises,” “including/includes” and “having/has” as used in the claims are open languages and do not exclude unrecited elements. The term “or” generally covers “and/or”, unless otherwise specified. The terms “about” and “substantially” used throughout the specification and appended claims are used to describe and account for small fluctuations or slight changes that do not materially affect the nature of the invention.

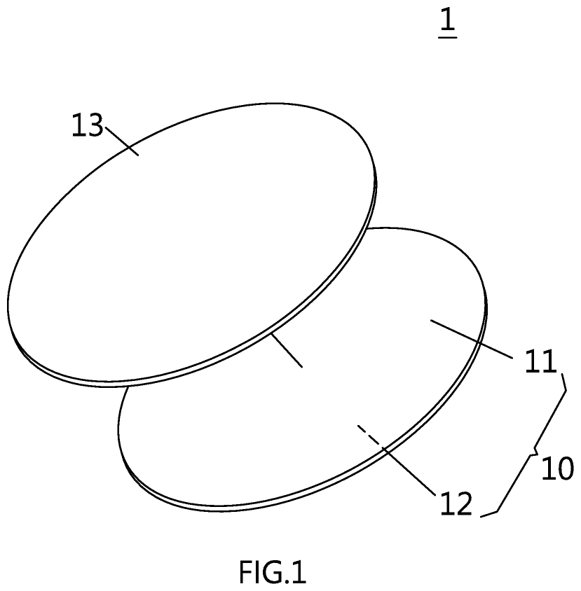

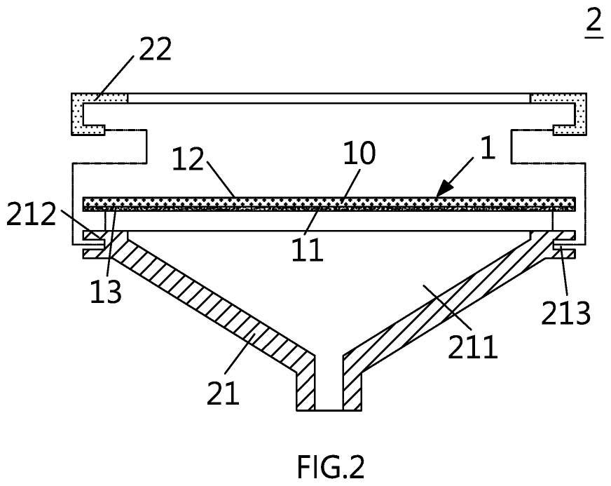

Referring to FIG. 1, a diaphragm 1 mainly comprises a plate-shaped vibratable portion 10 having a first surface 11 and a second surface 12 opposite to the first surface 11. The diaphragm 1 further comprises a compliant structural layer 13. The first surface 11 may be coated entirely with the compliant structural layer 13. Alternatively, the compliant structural layer 13 may be disposed on an outer periphery of the first surface. The diaphragm 1 according to the invention is adapted to being mounted on a sound collecting head for filling the gap between the diaphragm 1 and the sound collecting head, thereby improving the airtight sealing between the two parts. The sound collecting head can be applied to a stethoscope, a speaker, a hearing aid, and the like. According to the embodiment shown in FIG. 2 where the sound collecting head is used in a stethoscope, the stethoscope includes a sound receiving unit 2 for receiving sound, and a sound transmitting unit (not shown) connected to the sound receiving unit 2 for transmitting sound. The sound receiving unit 2 includes a sound collecting head 21 and the diaphragm 1. The sound transmitting unit has a conduit and an ear tube connected to the conduit, wherein the conduit is connected to the sound collecting head 21, and the ear tube is connected at one end with an earplug at an end thereof.

The sound collecting head 21 is configured in a substantially bell shape, thus defining a sound collecting chamber 211 having an opening where an annular lip portion 212 is disposed and protrudes outwardly from the sound collecting head 21. The diaphragm 1 is adapted for being brought in contact through the compliant structural layer 13 with and removably mounted on the lip portion 212, so that the diaphragm 1 seals the opening of the sound collecting chamber 211. While the appended drawings illustrate that the sound collecting head 21 is configured in a bell shape, the shape of the sound collecting head 21 may vary. For example, the sound collecting head 21 can be conical, cylindrical, polygonal, or multi-faceted. In a preferred embodiment, the diaphragm 1 is configured in a plate shape having an outer contour conforming in shape to the lip portion 212 of the sound collecting chamber 211. While the appended drawings illustrate that the diaphragm 1 is circular-shaped, the diaphragm 1 may have any two-dimensional shape known in the art (e.g., an elliptical shape). The shape of the diaphragm 1 may be modified to conform in shape to the opening of the sound collecting chamber 211 without compromising the sound quality and the ease of mounting the diaphragm 1.

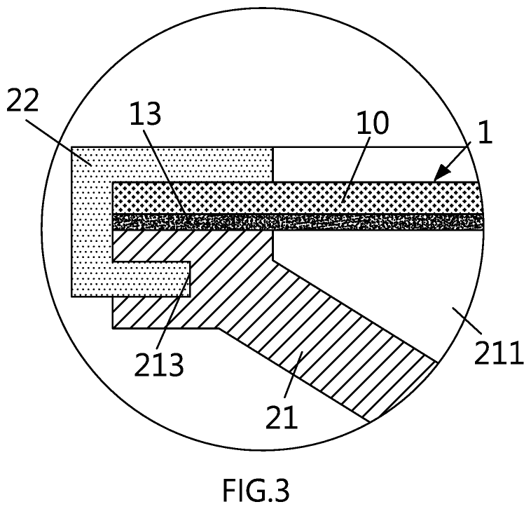

In a preferred embodiment, the invention further comprises a retaining ring 22 for clamping an outer periphery of the diaphragm 1 and the sound collecting head 21, so as to secure the diaphragm 1 to the sound collecting head 21. The sound collecting head 21 is formed with a groove 213 at the lip portion 212. The groove 213 is configured to be complementary in shape to the retaining ring 22, so that the retaining ring 22 may engage at one side with the groove 213 and abut at the other side against the second surface 12 of the diaphragm 1, thereby fastening the diaphragm 1 to the sound collecting head 21. Preferably, the retaining ring 22 is elastic, for example, being made of a resilient material such as thermoplastic polyurethanes, to provide the tension required to achieve a secure grip. By virtue of the structure above, the compliant structural layer 13 can be brought into contact with the sound collecting head 21. Now referring to FIG. 3, taking advantage of its compliance and elasticity characteristics, the compliant structural layer 13, after the diaphragm 1 is fastened to the sound collecting head 21 by the retaining ring 22, is slightly deformed to fill the gap between the diaphragm 1 and the sound collecting head 21 to increase the airtight sealing between the two parts, so that an airtight space is formed between the diaphragm 1 and the sound collecting chamber 211. As a result, the sound attenuation rate and the loss of acoustic vibration energy are greatly reduced, and the auscultation quality is improved. Meanwhile, the diaphragm 1 can hardly be displaced relative to the sound collecting head 21 by an external force after positioning.

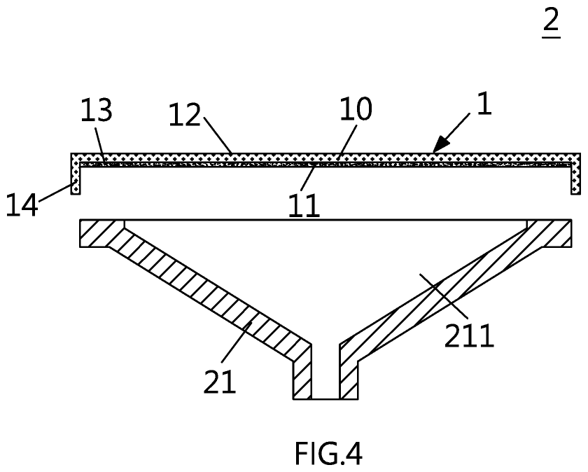

According to another preferred embodiment shown in FIG. 4, the vibratable portion 10 has an outer periphery protruding from the first surface 11 to constitute a retaining portion 14, and the retaining portion 14 is used to replace the above-mentioned retaining ring 22 for securing the diaphragm 1 to the sound collecting head 21. The retaining portion 14 is integrally formed with the vibratable portion 10, so that the diaphragm 1 can be quickly assembled on the sound collecting head 21 without other parts, and the user can even disassemble and assemble with one hand by himself. The user can decide to disassemble and replace the diaphragm depending upon the number of times of use, the state of the patient or the hygienic condition of the external environment, thereby effectively reducing indirect bacterial and viral infection, and meeting medical and hygienic requirements.

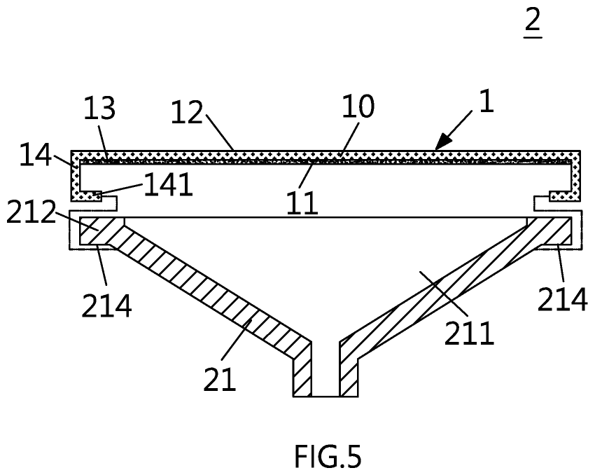

According to another preferred embodiment shown in FIG. 5, the retaining portion 14 is further bent towards an inner side of the vibratable portion 10 to form a flange 141. The sound collecting head 21 is formed with an abutting wall 214 on a bottom side of the lip portion 212 for engaging and positioning the flange 141, and the diaphragm 1 is adapted for being fastened to the sound collecting head 21. In this embodiment, the flange 141 is used to abut against the abutting wall 214 to achieve firm fixation of the diaphragm 1 to the sound collecting head 21.

The diaphragm 1 can be made of any material known in the art that is suitable for use as a stethoscope diaphragm. Examples of the material include, but are not limited to, resins (e.g., polycarbonates, polyethylene terephthalates, and glass fiber epoxy resins) and metals (e.g., stainless steel). A suitable thickness of the diaphragm 1 is between about 0.15 mm and 0.35 mm, preferably between about 0.15 mm and 0.22 mm. The compliant structural layer 13 may be made of a compliant material. The term “compliant material” as used herein may refer to a material which is relatively more elastic than the vibratable portion 10. In some embodiments, the compliant material may refer to a material that is capable of undergoing a reversible deformation responsive to the pressure generated by assembly with the sound collecting head. The compliant material includes, but is not limited to, silicones, soft touch paints, thermoplastic polyurethanes (TPU) and thermoplastic rubbers (TPR). A suitable thickness for the compliant structural layer 13 is between about 0.04 mm and 0.1 mm, preferably between about 0.04 mm and 0.08 mm. The compliant structural layer 13 may be built up by spraying, injection molding, in-mold injection, etc. In a preferred embodiment, a liquid compliant material is applied to cover the first surface 11 of the diaphragm 1 by spraying and then cured into the compliant structural layer 13 via a curing step; wherein a processing temperature of the curing step is between 45° C. and 50° C.

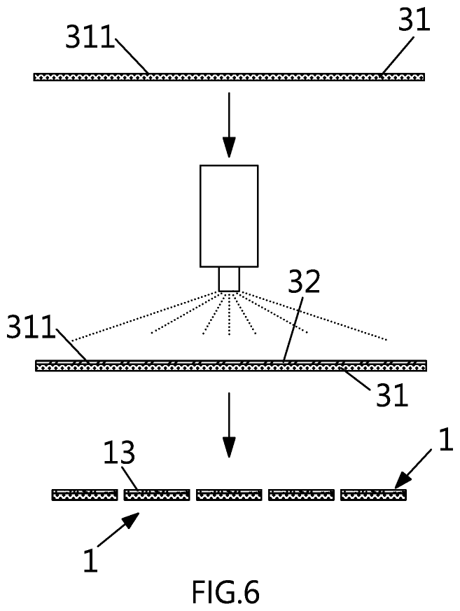

According the manufacturing flowchart shown in FIG. 6 where the diaphragm 1 is configured in the form of a flat plate, a plate-shaped membrane 31 having a first surface 311 is provided. The first surface 311 of the membrane 31 is sprayed with a compliant material 32 which is then subjected to curing, so that it is cured to form the compliant structural layer 13. Finally, the membrane 31 coated with the compliant structural layer 13 is stamped into a plurality of diaphragms 1 coated with the compliant structural layer 13 and having an outer contour conforming in shape to the opening of the sound collecting chamber 211.

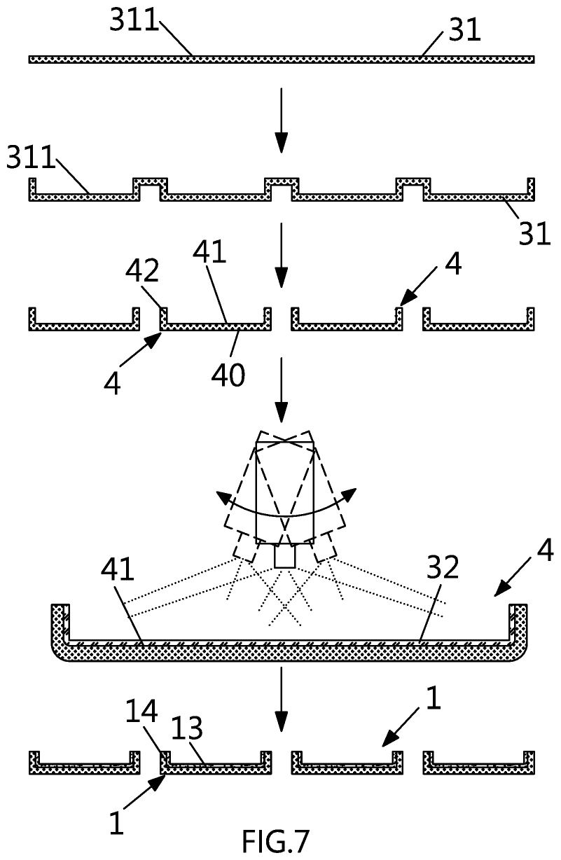

According an alternative embodiment shown in FIG. 7, a plate-shaped membrane 31 is provided. The membrane 31 is subjected to a preforming step and a stamping step to form a plurality of semi-finished diaphragms 4, each having a plate-shaped vibratable portion 40 having the first surface 311 and a retaining portion 42 extending from an outer periphery of the vibratable portion 40 in such a manner protruding from and substantially perpendicular to a first surface 41. The first surfaces 41 of the semi-finished diaphragms 4 are then sprayed with the compliant material 32. Finally, the compliant material 32 is subjected to curing, so that the compliant material 32 is cured into the compliant structural layer 13 to obtain a plurality of diaphragms 1 provided with the compliant structural layer 13 and the retaining portion 14.

In the embodiment where the compliant structural layer 13 is partially disposed at the outer periphery of the first surface 11, the compliant structural layer 13 may be configured in the form of a continuous annular ring or alternatively a discontinuous ring composed of several arc compliant structural layers 13 arranged on the first surface 11, both conforming in shape to the sound collecting head 21. In the embodiment where the vibratable portion 10 is provided with the retaining portion 14 and the compliant structural layer 13 is disposed on the entire first surface 11, the spraying may be performed by a four-axis dispensing device, so that the compliant material 32 can be applied on the inner side of the retaining portion 14 by rotation, thereby enhancing the convenience of processing and improving the productivity.

The following examples are provided for illustrative purpose rather than limiting the scope of the invention.

### Example 1: Planar Diaphragm

The diaphragm was produced by the steps of: providing a planar membrane having a thickness from 0.175 mm to 0.178 mm and of a material of polycarbonate; spraying a silicone rubber (i.e., a compliant material) on a first surface of the membrane; and curing the compliant material to form a compliant structural layer having a thickness from 0.042 mm to 0.045 mm. The diaphragm having a thickness of 0.22 mm was thus obtained and then secured to a sound collecting head of a stethoscope by a retaining ring.

### Example 2: Diaphragm with Retaining Portion

The diaphragm was produced by the steps of: providing a membrane having a thickness from 0.175 mm to 0.178 mm and of a material of polycarbonate; preforming and stamping the membrane into semi-finished diaphragms, each having a retaining portion; spraying a silicone rubber (i.e., a compliant material) on the respective first surfaces of the semi-finished diaphragms; and curing the compliant material to form a compliant structural layer having a thickness from 0.042 mm to 0.045 mm. The diaphragm provided with the retaining portion and having an overall thickness of 0.22 mm was thus obtained. It is adapted for direct assembly with a sound collecting head of a stethoscope.

### Comparative Example 1: Comparison with Commercially Available Counterpart

The diaphragms obtained in Examples 1 and 2, as well as a commercially available diaphragm (Littmann® Master Classic II™ Minnesota Mining and Manufacturing Company), were used for assembly with the sound collecting head of a stethoscope (model TS-2000), respectively.

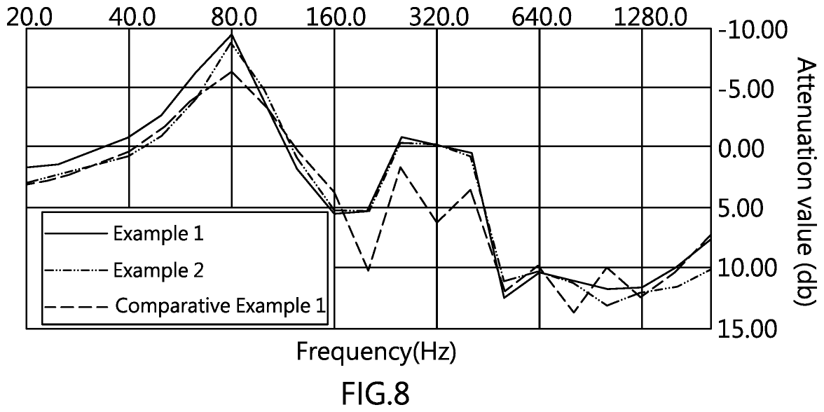

The frequency responses of the assembled stethoscopes were measured. The results are shown in Table 1 and FIG. 8.

TABLE 1
Attenuation values (db) at different frequencies (Hz)

| | Attenuation Values (db) | | |
|---|---|---|---|
| Frequency (Hz) | Example 1 | Example 2 | Comparative Example 1 |
| 20 | 1.70 | 3.00 | 3.10 |
| 25 | 1.40 | 2.40 | 2.60 |
| 40 | −0.80 | 0.70 | 0.35 |
| 50 | −2.70 | −0.90 | −1.5 |
| 63 | −6.20 | −4.10 | −4.25 |
| 80 | −9.40 | −8.80 | −6.30 |
| 100 | −3.70 | −4.90 | −3.65 |
| 200 | 5.30 | 5.30 | 10.25 |
| 250 | −0.90 | −0.40 | 1.65 |
| 315 | −0.20 | −0.30 | 6.35 |
| 400 | 0.40 | 0.7 | 3.45 |
| 500 | 12.60 | 11.10 | 12.05 |
| 630 | 10.40 | 10.30 | 9.80 |
| 800 | 11.20 | 11.30 | 13.85 |
| 1000 | 11.80 | 13.10 | 10.00 |

As shown in Table 1 and FIG. 8, within certain frequency ranges, especially the auscultation frequency ranges commonly used by medical care personnel, such as the first heart sound (40 Hz to 60 Hz), the second heart sound (60 Hz to 100 Hz), the third heart sound (10 Hz to 20 Hz), the fourth heart sound (20 Hz to 30 Hz) and the lung sound (200 Hz to 1000 Hz), the diaphragms disclosed herein (i.e., those obtained in Example 1 and 2) exhibited relatively low attenuation values at frequencies from 20 Hz to 100 Hz and from 160 Hz to 480 Hz, as compared to the commercially available product. The results indicate that the diaphragms coated with the compliant structural layer as disclosed herein can prevent the acoustic vibration energy from dissipating and improve the auscultation quality.

While the invention has been described with reference to the preferred embodiments above, it should be recognized that the preferred embodiments are given for the purpose of illustration only and are not intended to limit the scope of the present invention and that various modifications and 5 changes, which will be apparent to those skilled in the relevant art, may be made without departing from the spirit and scope of the invention.

1. A diaphragm, comprising:
a vibratable portion having a first surface;
a retaining portion integrated with the vibratable portion and protruding from the first surface along an entire outer periphery of the vibratable portion; and
a compliant structural layer disposed on the first surface.

2. (canceled)

3. The diaphragm according to claim 1, wherein the thickness of the compliant structural layer is between 0.04 mm and 0.08 mm.

4. The diaphragm according to claim 1, wherein the first surface is coated entirely with the compliant structural layer.

5. The diaphragm according to claim 1, wherein the compliant structural layer is disposed on an outer periphery of the first surface.

6. The diaphragm according to claim 1, wherein the compliant structural layer is formed by curing a compliant material selected from the group consisting of silicones, soft touch paints, thermoplastic polyurethanes and thermoplastic rubbers.

7. (canceled)

8. The diaphragm according to claim 1, wherein the retaining portion is further bent inwardly to constitute a flange.

9. A stethoscope, comprising:
a sound receiving unit for receiving sound, comprising a sound collecting head and a diaphragm, wherein the diaphragm comprises a vibratable portion having a first surface and a compliant structural layer disposed on the first surface, with the compliant structural layer of the diaphragm being arranged to face and attached to the sound collecting head; and
a sound transmitting unit connected to the sound receiving unit for transmitting the sound.

10. (canceled)

11. The stethoscope according to claim 9, wherein the thickness of the compliant structural layer is between 0.04 mm and 0.08 mm.

12. The stethoscope according to claim 9, wherein the first surface is coated entirely with the compliant structural layer.

13. The stethoscope according to claim 9, wherein the compliant structural layer is disposed on an outer periphery of the first surface.

14. The stethoscope according to claim 9, wherein the compliant structural layer is formed by curing a compliant material selected from the group consisting of silicones, soft touch paints, thermoplastic polyurethanes and thermoplastic rubbers.

15. The stethoscope according to claim 9, further comprising a retaining ring for clamping an outer periphery of the diaphragm and the sound collecting head, so as to retain the diaphragm on the sound collecting head.

16. The stethoscope according to claim 15, wherein the sound collecting head is formed with a groove that is configured to be complementary in shape to the retaining ring.

17. A stethoscope, comprising:
a sound receiving unit for receiving sound, comprising a sound collecting head and a diaphragm, wherein the diaphragm comprises a vibratable portion having a first surface, a compliant structural layer disposed on the first surface, and a retaining portion extending from an outer periphery of the vibratable portion and protruding from the first surface, with the compliant structural layer of the diaphragm being arranged to face and attached to the sound collecting head and the diaphragm being secured through the retaining portion to the sound collecting head; and
a sound transmitting unit connected to the sound receiving unit for transmitting the sound.

18. (canceled)

19. The stethoscope according to claim 17, wherein the thickness of the compliant structural layer is between 0.04 mm and 0.08 mm.

20. The stethoscope according to claim 17, wherein the first surface is coated entirely with the compliant structural layer.

21. The stethoscope according to claim 17, wherein the compliant structural layer is disposed on an outer periphery of the first surface.

22. The stethoscope according to claim 17, wherein the compliant structural layer is formed by curing a compliant material selected from the group consisting of silicones, soft touch paints, thermoplastic polyurethanes and thermoplastic rubbers.

23. The stethoscope according to claim 22, wherein the retaining portion is further bent inwardly to constitute a flange.

24-36. (canceled)
